(19) United States
(12) Patent Application Publication
(10) Pub. No.: US 2003/0213501 A1
(43) Pub. Date: Nov. 20, 2003

(54) Apparatus and method for cleaning an endoscope

(76) Inventors: Timothy Thomson, West Newbury, MA (US); Dan H. Stoddard, Kennebunk, ME (US)

Correspondence Address: Barry Evans, Esq., Kramer Levin Naftalis & Frankel LLP, 919 Third Avenue, NEW YORK, NY, 10022, US

(21) Appl. No.: 10/395,931

(22) Filed: Mar. 24, 2003

Related U.S. Application Data
(60) Provisional application No. 60/370,125, filed on Apr. 6, 2002.

(51) Int. Cl. B08B 9/04

(52) U.S. Cl. 134/8; 15/104.16; 15/104.2; 15/104.94; 300/21

(57) ABSTRACT

The invention is broadly in a scrubber for cleaning the lumen of a cannula of an endoscopic medical instrument comprising:

(a) an elongate, axial structure having a multiplicity of scrubber elements, e.g. bristles, along the length thereof, said scrubber having a length sufficient to extend through at least a portion of said lumen,

(b) a layer of hydrophilic polyurethane deposited on said scrubber elements in an amount effective to absorb a solution of detergent and/or enzyme solution; and

(c) a solution of detergent and enzyme absorbed in said layer of hydrophilic polyurethane,

said scrubber having a cross section of a size and configuration to closely fit within, engage and mechanically contact the walls of said lumen. The scrubber is used to clean the lumen of the cannula of medical instrument by degrading, dispersing and dissolving said debris and mechanically dislodging it from said lumen.

### FIELD OF THE INVENTION

The invention relates to an apparatus and method for scrubbing and cleaning cannulated medical instruments using an elongated scrubbing member coated with hydrophilic polyurethane containing agents which degrade, disperse and dissolve biological materials.

### BACKGROUND OF THE INVENTION

Surgery, diagnosis and other medical procedures are frequently performed using endoscopes and other elongated instruments which include narrow, elongated housings or cannulas through which fiber optic cables, rods, and other surgical implements are inserted or through which fluids are withdrawn. Endoscopes and other cannulated instruments are inserted through small surgical openings or natural mucosa in the body, thus reducing the invasiveness of a surgical or diagnostic procedure. Endoscopes are representative of such instruments as described herein, but all such instruments are included in the group to which the invention is addressed. Tools on the distal end of the endoscope, inside the body, are manipulated by squeezing scissors-like handles and other such mechanisms located on the proximal end of the endoscope, outside the body. Endoscopes for insertion into mucosa are flexible. Surgical endoscopes have a rigid outer covering and a rod which moves within the covering to control the surgical tool located on the distal end.

During procedures, debris, e.g., body fluids, blood, feces, pus and the like, is drawn into the narrow, elongated cannulas, especially when control rods and cables move within them. Since endoscopes are reused, if they are not thoroughly cleaned prior to disinfection or sterilization such debris can pass through those stages intact and can be introduced into another patient, leading to infection or other complications. Possible transmission of bacterial, viral, and mycobacterial infections can occur.

Studies have shown that many gastrointestinal endoscopes presumed ready for us in patients contained biological materials. For example, hepatitis C virus has been found in endoscopes following upper gastrointestinal endoscopy. Accordingly, a need exists for an effective way to remove biological debris from the cannulas and completely clean them.

The complexities of endoscopes complicate the cleaning procedure. Reusable instruments are not necessarily designed with a view to the ease of cleaning them. Residual bioburden remaining after cleaning, on and in narrow channeled endoscopes, is known to be a source of patient to patient disease transmission. Scrubbing every channel is thus mandatory in order to adequately clean the endoscope.

The devices presently used for cleaning debris from interior channels of endoscopes include bristle brushes and enzymatic soaps. These are of limited effectiveness. In techniques used in hospitals today the used endoscopes are soaked in tubs containing a detergent and an enzyme. The combined action of the detergent and enzyme loosens the debris within the cannulas of the endoscopes. A brush, which typically comprises an elongated handle and a short length of bristles, commonly less than one inch, is then inserted into the lumen of the cannula and moved about to brush the inside surface of the lumen.

This method is of limited effectiveness because the debris remains caked upon the interior surfaces of the lumen. The endoscopes are then put through a high-level disinfection process or sterilized in a sterilization chamber. Where deposits of debris have been left on the interior surfaces of the cannula, these deposits may become hardened and crosslinked. While the outer surface of such deposit may become sterilized, the protected underparts may be still rife with live bacteria. It is these live bacteria which can cause extraordinary pathological problems if dislodged when the endoscope is used on the next patient.

Valentine et al., U.S. Pat. No. 5,382,297, and Cercone et at., U.S. Pat. No. 5,274,874, describe apparatus and methods for cleaning endoscopes using, for example, a sponge impregnated with a composition of water, glycol, and a water-soluble wetting agent. The impregnated sponge is packaged in a container designed to facilitate cleaning of the endoscope.

Jackson, U.S. Pat. No. 4,517,702, relates to a sponge which is used to clean and sterilize endoscopes.

Bowman et al., U.S. Pat. No. 5,755,894, describes a hand-operated apparatus for forcing a cleaning solution through an endoscope to remove surgical debris. This device employs a syringe or other source of pressure to force a cleaning solution through the cannula of an endoscope. The device cleans debris from the cannula by flushing it away. This method is not particularly effective.

With the increasing demand for endoscopy, it is critical to use effective protocols for cleaning instruments. To date there are no nationally agreed upon standards for these products.

### OBJECTS OF THE INVENTION

It is a primary object of the invention to improve the effectiveness of apparatus and methods for cleaning the cannulas of endoscopes.

It is further a related object of the invention to provide means which effectively remove the surgical debris within the cannula of an endoscope that has been used in a surgical procedure.

It is still a further object of the invention to provide an improved scrubber which mechanically contacts the surfaces of the lumen of the cannulas and simultaneously delivers agents which degrade, disperse or dissolve the biological debris from those surfaces.

It is a further object of the invention to provide a scrubber which performs these functions at low costs.

It is yet another object of the invention to provide methods for a manufacture of the scrubber of the invention.

### SUMMARY OF THE INVENTION

The invention is broadly in a scrubber for cleaning the lumen of a cannula of an endoscopic medical instrument comprising:

(a) an elongate, axial structure having a multiplicity of scrubber elements, e.g. bristles, along the length thereof,

(b) a layer of hydrophilic polyurethane deposited on said scrubber elements in an amount effective to absorb a solution of detergent and/or enzyme solution; and

(c) a solution of detergent and/or enzyme absorbed in said layer of hydrophilic polyurethane,

said scrubber having a cross section of a size and configuration to closely fit within, engage and mechanically contact the walls of said lumen.

The apparatus is capable of removing biological debris from the cannula of an endoscope. Agents selected to cleanse and/or degrade, disperse or dissolve debris in the cannula, such as detergents and enzymes, are imbibed into the polyurethane. The apparatus is wetted to activate the impregnated enzyme and detergent and to form an active cleansing solution. The apparatus is then inserted into the cannula. A solution is formed that maintains contact with the entire surface of the lumen of the cannula during the cleaning process. The cannula is scrubbed for a time sufficient to permit the solution to uniformly contact the cannula and to the degrade, disperse or dissolve debris and then to mechanically dislodge and remove it.

The scrubbers of the invention can deliver their enzymes and detergents into the biopsy channel, suction and air lumens of endoscopes. In their various sizes and configurations, the scrubbers in those channels maintain direct intraluminal contact. In this way they target common biocontaminants of endoscopes and lumens. They provide a safe, fast and most importantly, a convenient way of removing gross biomaterial from the channels of endoscopes used in modern medicine.

While it is not the purpose of the invention to sterilize endoscopic instruments and no claim is made that they sterilize or completely decontaminate such instruments, in practice the scrubbers of the invention are capable of removing substantially all of the biological debris from the cannulae of the instruments and rendering the instruments amenable to complete disinfection or sterilization by other procedures.

The invention is also in a method for the manufacture of a scrubber, e.g. a brush, for cleaning the passageways of the cannula of an endoscopic instrument comprising: coating an elongate axial structural member having a multiplicity of scrubber elements, e.g. bristles, along the length thereof with a layer of hydrophilic polyurethane and absorbing in said layer of hydrophilic polyurethane an amount of solution of detergent and/or enzyme effective to clean the surfaces of the lumen or cannula of said endoscopic instrument upon scrubbing contact therewith, by degrading, dispersing or dissolving and mechanically removing said biological components.

### BRIEF DESCRIPTION OF THE DRAWINGS

FIG. 1 is a side view of a rigid endoscope which can be cleaned with the scrubbers of the invention.

FIG. 2 is a photograph of a pipe cleaner which can be coated with hydrophilic polyurethane to make a brush-like scrubber according to the invention.

FIG. 3 is a photograph of a brush-like scrubber coated with hydrophilic polyurethane according to the invention.

FIG. 4 is a schematic view of a production line for making the coated scrubbers of the invention.

### DETAILED DESCRIPTION OF THE INVENTION

In its broadest embodiment, the invention is for cleaning the cannula of a medical instrument. The invention comprises:

(a) an elongate scrubbing member having a multiplicity of scrubber elements along the length thereof, and

(b) a layer of hydrophilic polyurethane deposited on said scrubbing member,

said scrubbing member having a cross section of a size and geometry adapted to fit within and mechanically engage the inner surfaces of the lumen of said cannula.

In a further broad embodiment, the scrubbing member comprises elements (a) and (b) and the layer of hydrophilic polyurethane contains a solution of detergent and/or enzyme in an amount effective to create a solution within the cannula of the instrument which cleanses it and/or degrades, disperses and dissolves the biological materials therein.

The term scrubbing member is used in the broadest sense. It includes any more or less axial structure comprised of any material which is capable of providing both support for a layer of hydrophilic polyurethane and sufficient mechanical strength and integrity so that the coated member will physically contact the surfaces of the cannula, deliver the solution of detergent and/or enzyme so as to cleanse degrade, disperse or dissolve biological debris, and then dislodge that debris from the cannula.

The scrubbing members may be comprised of metal, plastic or fabric or any other material capable of supporting the coating of hydrophilic polyurethane and capable of providing either directly, or, indirectly through the layer of polyurethane, the requisite contact with the surfaces of the lumen of the cannula of the endoscope. The scrubbing member thus may be a swab, a brush, a mop etc. and all are within the scope of the invention if capable of performing the dual functions described above. The scrubbing member may thus be a metal, or plastic wire configured to retain the coating of hydrophilic polyurethane or may be a braided or unbraided rope, string, yam filament or other configuration of fabric capable of taking the overcoat of hydrophilic polyurethane and providing the lateral rigidity to directly or indirectly engage the surfaces of the lumen of the cannula of the instrument. A preferred embodiment, described more fully below, includes a braided wire with synthetic bristles, e.g. a pipe cleaner, which is overcoated with hydrophilic polyurethane.

Thus the invention is a scrubber which comprises:

(a) an elongate brush comprising an axial structural member and a multiplicity of bristles extending outwardly therefrom along the length of said axial member;

(b) a layer of hydrophilic polyurethane deposited on said bristles in an amount from 0.10 to 3.0 grams per meter of said brush and effective to absorb an amount of detergent and enzyme solution, and

(c) a solution of detergent and enzyme absorbed in said layer of hydrophilic polyurethane,

said scrubber having a cross section of a size and configuration to closely fit within and mechanically engage and contact the walls of said lumen.

In a preferred embodiment, the scrubbing member may have a length sufficient to extend through at least a substantial portion of said cannula.

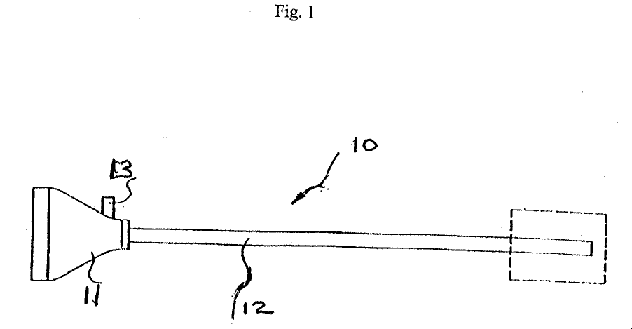

With reference to FIG. 1, an endoscope 10 is shown with a main body 11 and a cannula 12 distally connected thereto. Also shown is a connection 13 by which a light guide, which is not shown in FIG. 1, is inserted into the cannula 12. An eyepiece of an interface for appropriate observation devices is generally provided in main body 11. These components are known in the art and are not shown in detail.

The scrubbers of the invention are adapted to be manually or automatically rubbed or brushed against the interior surfaces of the cannula of a medical instrument, e.g. an endoscope. While the invention has been described as relating to the cannula of an endoscope, it will be understood by those skilled in the art that the invention is adaptable for use in any medical instrument having an interior portion which must be accessed by a cleaning device. The term cannula has been used as a description of a preferred embodiment and is not limited. Similarly, the invention can be used with any medical instrument where interior portions must be cleaned of biological debris.

The cross section of the scrubber can be of any suitable geometry, e.g. round, square, triangular or the like in order to closely match the lumen of the cannula. What is of importance is that during use the surface of the scrubber come in close proximity and contact with the interior surfaces of the lumen so that the scrubber, whether or not carrying enzymes and detergents, deposits such enzymes and detergents uniformly and exerts maximum action against those surfaces, loosens debris and permits that debris to be removed. The scrubbers can be made in various diameters, effective diameters and lengths, to match various instruments geometries.

Preferred scrubbers of the invention comprise brushes containing a multiplicity of bristles. In certain embodiments the bristles extend along the entire length of the brush, except for suitable handle or leader sections on one or both ends thereof. In other embodiments the bristles may extend along only a portion, e.g. 50% of the length of the brush but preferably along at least 75% and even more preferably along at least 90% of the length of the brush. By extending the bristles throughout the entire elongate brush, maximum efficiencies are achieved and the brush will more consistently contact all parts of the interior surfaces of the lumen of the cannula when it is used. Brushes may be long or short in length, e.g. from as little as an inch or two in length to as much as several feet in length. All such lengths are within the scope of the invention.

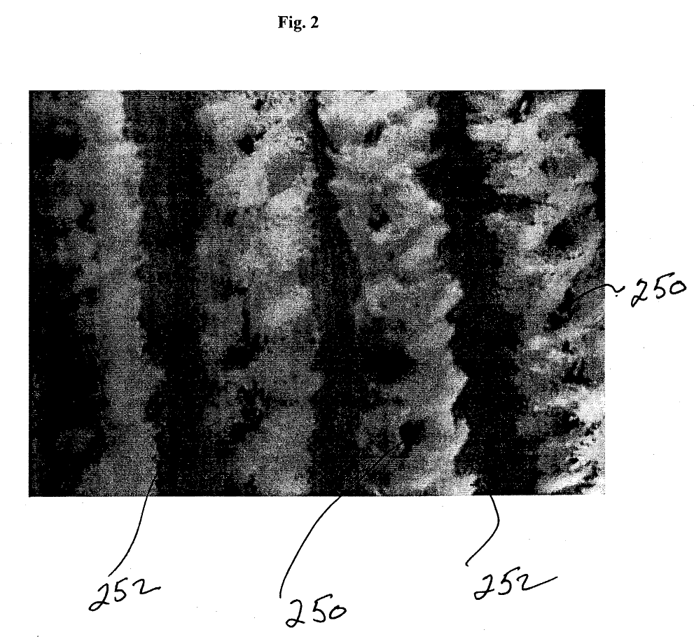

The density of the bristles is a matter of choice. With reference to FIG. 2, a preferred starting point for the production of a scrubber according to the invention, is a standard pipe cleaner. The pipe cleaner comprises an elongate axial member which may be a twisted pair of wires, preferably stainless steel (e.g. Type 304), which carry a multiplicity of bristles which extend outwardly from the axial member. The axial member may be stiff or flexible such that the brush can be passed through the cannula of the endoscope and moved back and forth.

In exemplary embodiments, the pipe cleaners used to make the brushes of the invention may have the dimensions and characteristics described below:

| Diameter of | | |
|---|---|---|
| Brush (mm) | Axial Member | Bristles |
| 1.5 | twisted pair of 0.12 and | one strand of polyester yarn |
| | 0.14 mm stainless steel | |
| | wire | |
| 2.5 | same as above | two strands of polyester yarn |
| 4 | twisted pair of 0.14 mm | three strands of polyester yarn |
| | stainless steel wire | |
| 6 | twisted pair of 0.14 mm | three strands of polyester yarn |
| | and 0.16 mm stainless steel | |
| | wire | |

The material of the bristles is likewise a matter of choice. Among the preferred bristles are those made of polyester or other polymer, woven cellulose, etc. Polyester bristles are preferred over cotton bristles because cotton bristles tend to mat down with the result that brushing action is less effective. Depending on the nature of the cleaning operation to be performed the bristles can be more or less dense along the length of the brush.

Hydrophilic polyurethanes are well known in art. Hydrophilic prepolymers suitable for use in the present invention, include, for example, isocyanate-capped polyether prepolymers with an NCO functionality of greater than 5% as more particularly described below. The prepolymers are based on polyether polyols capped with aromatic isocyanates such as, for example, toluene diisocyanate (TDI) or methylene diphenyl isocyanate (MDI) or with aliphatic isocyanate (HMDI). The polyether polyols are hydrophilic polyoxyalkylenes with a minimum of 40 mole % ethylene oxide.

Isocyanate-capped polyether prepolymers which have been found to be suitable for use in the practice of the present invention include, without limitation, prepolymers commercially available from the following companies: Lendell Manufacturing, St. Charles, Mich., U.S.A.; Rynel Ltd. Inc. Boothbay, Me., U.S.A.; Dow Chemical, Midland, Mich., U.S.A.; and Mace Adhesives and Coatings, Dudley, Mass., U.S.A. Table 1 below lists exemplary polyurethane prepolymers of these companies and the NCO functionality content of their respective prepolymers.

The polyurethane is coated on the elongated bristled brush in an amount of broadly from 0.10 to 3.00 grams per meter of brush, preferably from 0.25 to 2.00 grams per meter of brush and even more preferably from 0.50 to 1.50 grams per meter of brush. Enough polyurethane must be coated on the brush to provide a satisfactory medium for the absorption of enzyme and/or detergent, in those embodiments where enzyme and/or detergent are to be absorbed, and, whether or not enzyme and/or detergent are to be absorbed, to provide strong scrubbing action. The amount of hydrophilic polyurethane will vary depending on the diameter of the brush, the greater diameters having substantially higher levels of hydrophilic polyurethane coating per unit of length. Thus, for brushes having diameters from 1.0 mm to 5.0 mm the hydrophilic polyurethane may preferably be coated in an amount from 0.60 to 1.00 gms/meter. For brushes having diameters of from 5 mm to 10.0 mm the polyurethane is coated in an amount from 1.00 gms to 3.00 gms/meter.

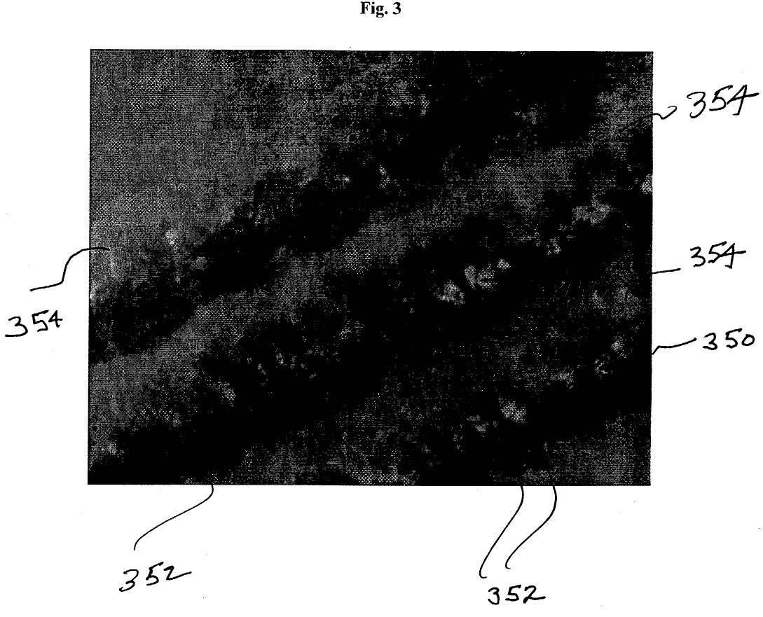

With reference to FIG. 2 and FIG. 3, the scrubber of the invention comprises the pipe cleaner as shown in FIG. 2, the bristles of which have been coated with a layer of hydrophilic polyurethane. The layer may extend in around and/or over the bristles, and the invention is not limited to any one or another disposition. Reference numerals 250 and 350 refer generally to the axial member. Reference numerals 252 and 352 refer to the bristles emanating outwardly from the axial member. Reference numerals 354 refer to the hydrophilic polyurethane coated on the bristles. As shown, the level of polyurethane coating may vary along the length of the brush provided that sufficient coating is present to carry out the objectives of the invention.

A detergent solution may be absorbed in the hydrophilic polyurethane. The detergent is preferably a non-ionic detergent such as Cal Foam ES 603 in aqueous solution. Other surface active agents can also be used. The solution contains broadly from 25 to 50% by weight of detergent, desirably 30 to 40% by weight and preferably 35 to 36% by weight. The solution is absorbed in the hydrophilic polyurethane in an amount broadly from 0.01 to 1.00 grams of solution per gram of the total coating i.e. the polyurethane and the detergent solution absorbed within it, preferably from 0.02 to 0.75 grams per gram of total coating and desirably from 0.05 to 0.50 grams per gram of total coating.

An agent capable of degrading, dispersing or dissolving biological contaminants, preferably an enzymatic solution containing proteases, lipases, and/or amylases, may be impregnated into the hydrophilic polyurethane. An exemplary enzymatic solution is “Enzymatic Cleaner” manufactured by Enzyme Solutions, Incorporated of Hickory, N.C. The enzymes are in a water solution containing broadly 1 to 10% by weight enzyme, desirably 1.0 to 5% by weight enzyme and preferably 1.5 to 3% by weight enzyme. The solution of enzyme can be used in broadly from 0.005 to 0.50 grams per gram of total coating, desirably from 0.01 to 0.30 grams per gram of total coating and preferably from 0.02 to 0.20 grams per gram of total coating.

### Methods of Making an Endoscope Scrubber

In an exemplary embodiment of methods of the invention for manufacturing a scrubber the bristled member is drawn through a vessel containing a prepolymer of hydrophilic polyurethane maintained within a temperature range of 45-50 degrees C. and then through an orifice in the underside of the vessel of a diameter such that excess prepolymer is removed and retained in the vessel. Alternatively, the bristled member may, after being drawn through the said vessel, be passed between two rotating rollers, each having a semi-curricular groove mating with the groove of the opposing roller so as to form an orifice of a diameter such that excess prepolymer is removed and returned to the vessel. The bristled member emerges from the vessel coated with a uniform, thin layer of prepolymer. It is immediately immersed in a second vessel containing water to initiate polymerization. Passage through the second vessel is calculated to provide a dwell time sufficient for water to be absorbed in an amount required to complete the polymerization reaction.

The bristled member is then drawn through a curing oven in which a temperature of 65-75 degrees C. is maintained. The polymerization reaction is completed and excess water is evaporated in this passage.

On exiting the curing oven, the bristled member has become a composite material of hydrophilic polyurethane foam bonded to and reinforced by the bristles of the bristled member. This composite material may at this point have its surface geometry modified to achieve a target diameter or surface texture by means of mechanical cutting, grinding, or abrading, e.g. the composite material may be passed through a series of grinders or carbide cutting wheels rotating at a speed of 30,000 rpm, such grinders or cutting wheels having contact surfaces describing the desired diameter and geometry.

The composite bristled member is then drawn through a third vessel where it absorbs agents to facilitate the removal, degradation, dispersion and dissolution of organic contaminants, including but not limited to such enzymatic agents as proteases and amylases, and detergents. The composite bristled member with imbibed solution is then drawn through a second drying oven or tunnel in which a temperature not exceeding the deactivation temperature of enzymes of 30-35° C. is maintained.

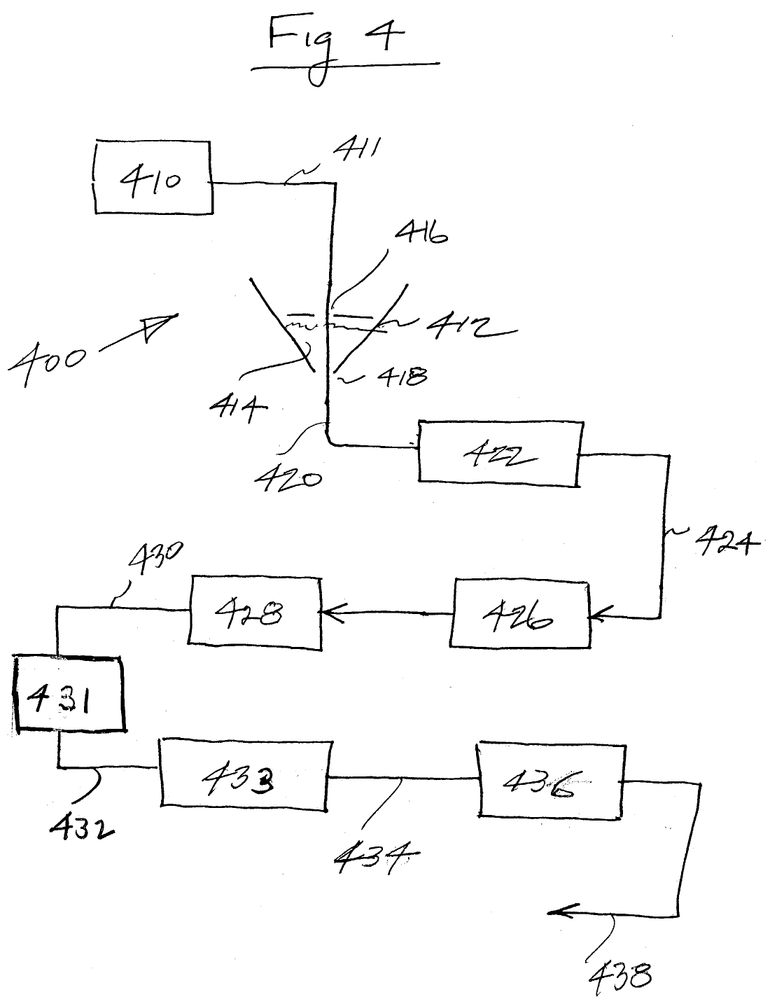

In FIG. 4, reference numeral 400 refers to a schematic diagram of a production line for making brushes of the invention. Reference numeral 410 refers to a source of elongated bristled member which may be for example a feed reel of pipe cleaner. The pipe cleaner is passed over feed rollers and the like, not shown, to hydrophilic polyurethane prepolymer application zone 412. Zone 412 may be any suitable vessel and contains a reservoir of prepolymer 414. The pipe cleaner passes through a first orifice 416 which is narrower in diameter than the nominal diameter of the pipe cleaner which is to be coated. The purpose of orifice 416 is to spread the bristles backward so is to achieve adequate coating of the bristles in reservoir 414. Preferably the diameter of orifice 416 is from 50-90% of the nominal diameter of the pipe cleaner and desirably it is from 60-80% of the diameter of the pipe cleaner. In certain preferred embodiments the diameter of orifice 416 is from 65-75% of the diameter of the pipe cleaner. Where small diameter pipe cleaners are being coated, e.g. these having a diameter of 1.5 mm to 2.5 mm, the first orifice may be omitted.

The pipe cleaner 411, in a brushed-back condition, then passes through reservoir 414 of polyurethane prepolymer wherein it is coated. The pipe cleaner then passes through the second orifice, reference numeral 418, which controls the amount of hydrophilic polyurethane which is coated on the pipe cleaner. By using the two orifices in application zone 412, one obtains an adequate and controlled amount of coating and avoids an annular coating where only the outer part of the bristles are coated leaving an uncoated area around the outer diameter of the axial member. The coated pipe cleaner is shown at 420.

Representative hydrophilic polyurethane prepolymers are described below in Table 1.

TABLE 1

| Rynel Prepolymer Type | % NCO |
|---|---|
| B-1 | 7.4-8.32 |
| A-62 | 10.0-11.4 |
| Trepol | 5.2-6.4 |
| Mace Adhesives & Coatings | |
| Bipol | 5.5-6.5 |
| Dow Chemical | |
| Hypol 2000 | 6.3-7.2 |
| Hypol 2002 | 6.3-7.2 |
| Hypol 3000 | 9.5-10.3 |
| Hypol 5000 | 9.5-11.13 |
| Lendell Manufacturing | |
| Prepol | 8.0-8.5 |

The method of analysis of the NCO functionality is described in Analytical Chemistry of Polyurethanes, Robert E. Krieger Publishing Company, Huntington, N.Y. (1979).

The prepolymer must be maintained at a viscosity broadly in the range from 3,000 to 20,000 cp, desirably in the range from 5,000 to 15,000 cp and most preferably in the range from 7,000 to 12,000 cp. This can be achieved by heating the prepolymer to a temperature broadly in the range from 32 to 49° C. before it is added to coating zone 412, or, by diluting the prepolymer in a suitable, nonreactive solvent such as acetone, to a dilution from 10 to 60% by weight prepolymer and preferably from 20 to 30% by weight prepolymer. In embodiments of the invention, solutions of prepolymer in acetone at a concentration from 10 to 20% by weight may be used.

The coated pipe cleaner 420 then passes to curing zone 422 where it passes through a water bath which causes the polymerization of the hydrophilic polyurethane.

The pipe cleaner having a coating of cured but wet hydrophilic polyurethane is shown at reference numeral 424. The coated pipe cleaner then passes to a drying zone 426 where it is contacted with a stream of compressed air to remove superficial water. It then passes to a heating chamber 428 wherein it is dried by infrared lamps or other suitable heat sources. It is critical to the proper operation of the production line, to remove most of the water from the coated polyurethane in other to achieve satisfactory absorption of the enzymes which are applied in subsequent steps. Thus the pipe cleaner having a coating of dried, cured polyurethane shown at reference numeral 430 should have less than 5% by weight water relative to the coating and preferably less then 1% by weight water relative to the coating.

The dried, coated pipe cleaner then passes through a series of grinders or carbide cutting wheels, 432. There the coated pipe cleaner contacts cutting wheels or grinders rotating at speeds up of to 30,000 rpm which shape them to the desired configuration. The shaped cleaner 432 then passes to enzyme and detergent bath 433. An aqueous solution of a detergent and enzymes is applied to the coated pipe cleaner in the amounts described. The pipe cleaner with absorbed detergent and enzyme solution, reference numeral 434, passes to a drier 436 where excess water is removed in a heated air stream. The temperature of heating zone 436 should not exceed 37° C. to avoid destruction of the enzyme. The finished product, reference numeral 438, desirably contains water in less than 5% by weight of the coating and preferably contains water in less than 1% by weight of the coating.

The finished product can be respooled for storage or cut to suitable lengths for its intended use. Short lengths of an axial member of the same nominal diameter as the brush and having some stiffness can be crimped to the ends of the cut lengths of brush to facilitate handling.

### Method of Using the Endoscope Brush

The scrubbers of the invention can be used in many ways which will be understood by those skilled in the art. For example, a brush consisting of the axial bristled member and hydrophilic polyurethane, but no absorbed detergent or enzyme, may be soaked in a bath of detergent and/or enzyme solution just prior to use, for a time sufficient to absorb an effective amount of enzyme and/or detergent, and then introduced into the cannula of the instrument as more fully described below.

A preferred method for using the brush involves cleaning the instrument channel of an endoscope (e.g., the cannula) by wetting a brush which contains enzyme and/or detergent absorbed in the hydrophilic polyurethane, inserting the apparatus into the cannula of the endoscope and rubbing the brush several times within the cannula to release the detergent and enzyme throughout the cannula. The apparatus is moved back and forth to physically detach the biological materials. The fluid is left in the cannula for a period of time sufficient to allow it to degrade, disperse and dissolve the debris present. The apparatus is then removed and the cannula may be flushed with water or other suitable cleaning fluid.

The apparatus of the invention may be used single ply or multiple ply as befits any particular endoscope or lumen configuration. The apparatus can also be used with or without soaking the endoscope in an enzyme/detergent bath.

Thus the brush illustrated in FIG. 3 is moistened and inserted lengthwise into cannula 12 of endoscope 10 (FIG. 1) at the distal or at the proximal end. Cannula 12 is scrubbed with the apparatus to elicit release of detergent and/or enzyme. The apparatus is left in cannula 12 for, e.g., 1 to 3 minutes to allow the enzyme to degrade proteins, fats, and other organic materials. Then, cannula 12 is scrubbed again to facilitate removal of degraded, dispersed and dissolved organic matter. The apparatus is then removed from cannula 12 and cannula 12 is flushed with water to expel the materials therein.

What is claimed is:

1. A scrubber for cleaning the cannula of a medical instrument comprising:
(a) an elongate scrubbing member having a multiplicity of scrubber elements along the length thereof, and
(b) a layer of hydrophilic polyurethane deposited on said scrubber elements, said scrubber having a cross section of a size and geometry adapted to fit within and engage the inner surfaces of the lumen of said cannula.

2. A scrubber for cleaning the cannula of a medical instrument comprising:
(a) an elongate brush having a length sufficient to extend through at least a substantial portion of said cannula, said brush including a multiplicity of bristles along the length thereof, and
(b) a layer of hydrophilic polyurethane deposited on said bristles, said brush having a cross section of a size and geometry adapted to fit within and engage the inner surfaces of the lumen of said cannula.

3. A scrubber for cleaning the cannula of a medical instrument comprising:
(a) an elongate scrubbing member having a multiplicity of scrubber elements along the length thereof,
(b) a layer of hydrophilic polyurethane deposited on said scrubber elements, and
(c) a solution of detergent and/or enzyme absorbed in said layer of hydrophilic polyurethane.

4. A brush for cleaning the cannula of a medical instrument comprising:
(a) an elongate brush having a length sufficient to extend through at least a substantial portion of said cannula, said brush including a multiplicity of bristles along the length thereof,
(b) a layer of hydrophilic polyurethane deposited on said bristles, and
(c) a solution of detergent and/or enzyme absorbed in said layer of hydrophilic polyurethane, said brush having a cross section of a size and geometry adapted to fit within and engage the inner surfaces of the lumen of said cannula.

5. A brush for cleaning the lumen of a cannula of an endoscopic surgical instrument comprising:
(a) an elongate brush comprising an axial structural member and a multiplicity of bristles extending outwardly therefrom along the length of said axial member,
(b) a layer of hydrophilic polyurethane deposited on said bristles in an amount from 0.10 to 3.00 grams per meter of said brush and effective to absorb a solution of detergent and/or enzyme, and
(c) a solution of detergent and enzyme absorbed in said layer of hydrophilic polyurethane,
said brush having a cross section of a size and configuration to closely fit within, engage and contact the walls of said lumen.

6. A brush as recited in claim 5 wherein said brush contains from 0.10 to 1.00 grams of detergent solution per gram of total coating and from 0.005 to 0.50 grams of enzyme per gram of total coating.

7. A method for the manufacture of a scrubber for cleaning the interior passageways of the cannula of an endoscopic medical instrument comprising: coating an elongate scrubbing member comprising an axial structural member and a multiplicity of scrubber elements along the length of said member with a layer of hydrophilic polyurethane.

8. A method for the manufacture of a brush for cleaning the lumen of a cannula of a medical instrument comprising: coating an elongate brush comprising an axial structural member and a multiplicity of bristles extending outwardly therefrom along the length of said member with a layer of hydrophilic polyurethane; and absorbing in said layer of hydrophilic polyurethane an amount of a solution of detergent and enzyme effective to clean, degrade, disperse and dissolve the surfaces of the lumen of the cannula upon brushing contact therewith.

9. A method for cleaning the cannula of a medical instrument comprising contacting the lumen thereof with a scrubber comprising:
(a) an elongate scrubbing member having a multiplicity of scrubber elements along the length thereof,
(b) a layer of hydrophilic polyurethane deposited on said scrubber elements, and
(c) a solution of detergent and/or enzyme absorbed in said layer of hydrophilic polyurethane.

10. A method for cleaning the cannula of a medical instrument comprising:
(a) placing a scrubber comprising
(i) and elongate scrubbing member having a multiplicity of scrubber elements along the length thereof, and
(ii) a layer of hydrophilic polyurethane deposited on said scrubber elements
in a solution of enzyme and/or detergent for a time sufficient to absorb an effective amount of enzyme and/or detergent in said layer of hydrophilic polyurethane; and
(b) introducing the scrubber of step (a) into the cannula of said medical instrument; and
(c) scrubbing the cannula of said instrument with said scrubber.
